(19) United States
(12) Patent Application Publication
(10) Pub. No.: US 2022/0149640 A1
(43) Pub. Date: May 12, 2022

(54) SMART CHARGING DEVICE WITH UV LAMP STERILIZATION AND ITS CONTROL METHOD

(71) Applicant: CHEN-SOURCE INC., Taoyuan (TW)

(72) Inventor: Wen-Cheng HSU, Taoyuan (TW)

(21) Appl. No.: 17/092,533

(22) Filed: Nov. 9, 2020

(51) Int. Cl. H02J 7/00; A61L 2/10; H05B 47/16; H05B 47/165

(52) U.S. Cl. CPC H02J 7/0044; A61L 2/10; H02J 7/0071; H02J 7/00304; A61L 2202/16; H05B 47/165; A61L 2202/11; A61L 2202/121; A61L 2202/122; H05B 47/16

(57) ABSTRACT

A smart charging device having a charging module and a UV lamp set mounted in the main body thereof is disclosed. The charging module includes power supply circuit, a power input unit connected to the power supply circuit for power, and a charging area connected to the power supply circuit for charging mobile devices. The UV lamp set includes a drive control circuit connected to the power supply circuit, a start unit and at least one UV lamp connected to the drive control circuit through the start unit. In addition to the charging function, the smart charging device also provides an ultraviolet sterilization function.

### BACKGROUND OF THE INVENTION

### 1. Field of the Invention

The present invention relates to charging device technology and more particularly, to a smart charging device with UV lamp sterilization and its control method. The smart charging device has a charging module and a UV lamp set mounted in the main body thereof. In addition to the function of charging mobile devices, the smart charging device is also designed with ultraviolet sterilization function to ensure the safety of use.

### 2. Description of the Related Art

With the rapid development of electronic technology and multimedia information, electronic devices such as smart phones, tablet computers, and notebook computers are developing toward light, thin, short, small, and powerful trends, mainly for small size, light weight, and convenient portability. Coupled with continuous innovation of soft and hardware functions, it is more flexible and practical in use. As the processing speed and computing performance of electronic devices continue to increase, their battery power is also depleted faster. In order to compensate for the charging of the batteries of the electronic devices, the environment (such as school, office or public place, etc.) where a large number of electronic devices is generally used is mostly equipped with fixed-point or mobile charging devices (such as charging carts or charging cabinets) to provide charging for multiple electronic devices.

In view of the above-mentioned use environment of charging carts or charging cabinets, most of them are used in schools or public places for specific personnel. Therefore, in the process of use, it is easy to cause bacteria, viruses and other pathogens to remain on the surface of the tablet or notebook computer. As a result, users are prone to mutual infection during the process of handling or touching with their hands, and cause safety hazards to human health. The traditional method of spraying or wiping with detergent or alcohol can achieve the functions of disinfection and sterilization, but if the user's hands accidentally come into contact with the detergent or alcohol, it is easy to cause skin irritation or chemical drug residues. After a while, it is difficult to reliably perform disinfection and sterilization due to negligence or fatigue.

Therefore, in today's era of climate change and globalization with many bacteria or viruses spread in the environment, how the industry can design a smart charging device that can charge electronic devices and provide a sterilization function to ensure safety in use is an important issue that needs to be improved by those engaged in this industry.

### SUMMARY OF THE INVENTION

The present invention has been accomplished under the circumstances in view. It is the main object of the present invention to provide a smart charging device with UV lamp sterilization, which has a charging module and a UV lamp set mounted in the main body (such as charging cart or charging cabinet) thereof. The charging module includes power supply circuit, a power input unit connected to the power supply circuit for power, and a charging area connected to the power supply circuit for charging mobile devices. The UV lamp set includes a drive control circuit connected to the power supply circuit, a start unit and at least one UV lamp connected to the drive control circuit through the start unit. In addition to the charging function, the smart charging device also provides an ultraviolet sterilization function. When the microcontroller of the drive control circuit detects that the safety start switch of the start unit has been activated, and when the door sensor switch detects that the door cover of the main body is closed, the power relay is controlled to provide power supply to the electronic ballast to activate the UV lamp sterilizing the storage mobile device. When the set sterilization time is reached, the UV lamp power supply will be turned off, so that the user will not easily cause mutual infection of the human body when taking or using the mobile device, which can ensure the safety of use.

According to another aspect of the present invention, the main body of the smart charging device can be a charging cart or charging cabinet. The drive control circuit of the UV lamp set detects whether the safety start switch of the start unit has been activated, and the indicator lamp on the lam press button switch will light up after activation, indicating that the current UV lamp sterilization function has entered the ready-to-start state. At this time, if the door cover of the main body is opened, the door sensor switch will not be activated, and the UV lamp will not be activated. This is a mechanism to prevent users from accidentally activating the sterilization function to prevent human body damage from UV leakage. Only after the door sensor switch is activated, the UV lamp will be activated to irradiate ultraviolet rays to the mobile device been placed in the main body to complete the action of disinfection and sterilization.

Other advantages and features of the present invention will be fully understood by reference to the following specification in conjunction with the accompanying drawings, in which like reference signs denote like components of structure.

### BRIEF DESCRIPTION OF THE DRAWINGS

FIG. 1 is an oblique top elevational view of a smart charging device with UV lamp sterilization in accordance with the present invention.

FIG. 2 is an exploded view of the smart charging device with UV lamp sterilization in accordance with the present invention.

FIG. 3 is a block diagram of the control circuit of the preferred embodiment of the present invention.

FIG. 4 is a flow chart of the UV lamp control method of the preferred embodiment of the present invention.

FIG. 5 is a schematic diagram of the present invention showing the door opened for placement of mobile devices.

FIG. 6 is a schematic diagram of the present invention showing the door closed to sterilize the mobile devices.

### DETAILED DESCRIPTION OF THE PREFERRED EMBODIMENT

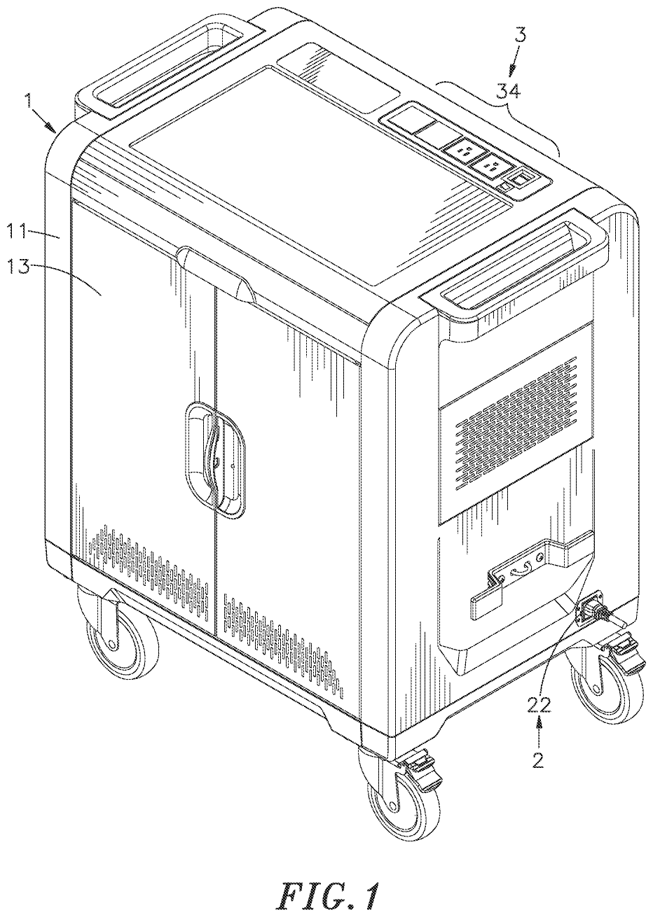

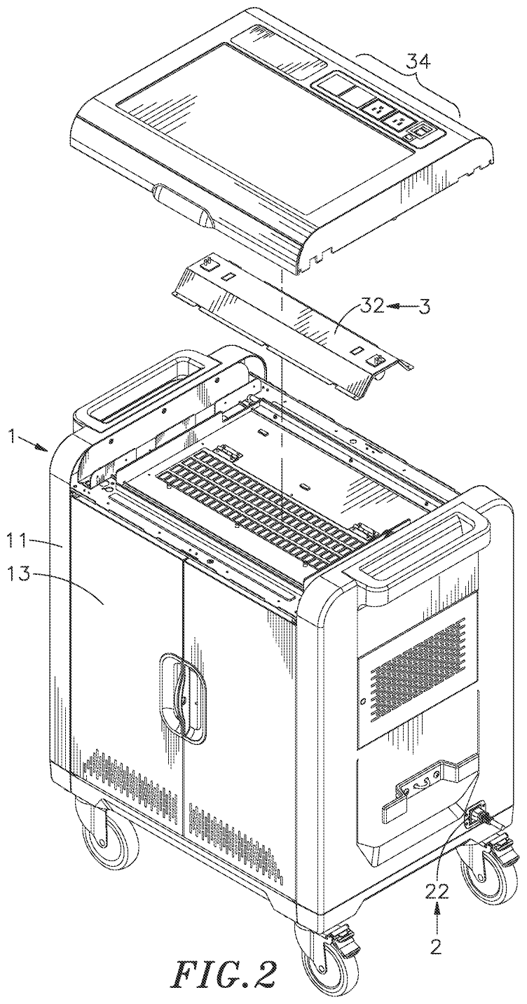

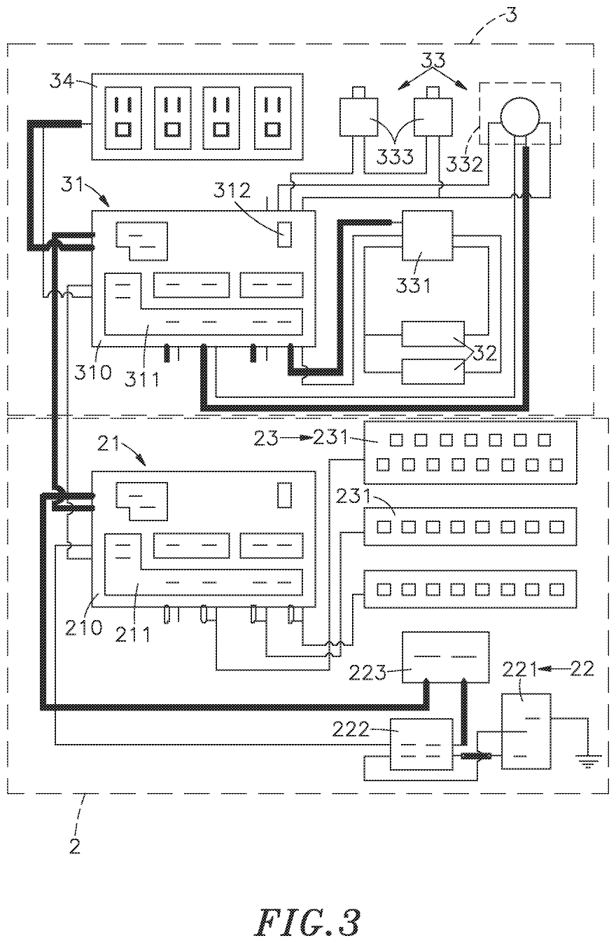

Referring to FIGS. 1-6, a smart charging device with UV lamp sterilization in accordance with the present invention is shown. The smart charging device with UV lamp sterilization comprises a main body 1, a charging module 2 and a UV lamp set 3.

The main body 1 is a housing 11 of rack or cabinet type. The housing 11 comprises a housing space 10, a plurality of partition plates 111 arranged in the housing space 10 at different elevations to divide the housing space 10 into a plurality of vertically spaced storage chambers 101, a carrier rack 12 mounted in each storage chamber 101 and providing a plurality of transversely spaced storage slots 121, and a door cover (such as double panel door, hinged door or sliding door) 13 installed on the front opening thereof.

The charging module 2 is installed in the housing space 10 of the housing 11 at a back side relative to the carrier rack 12, comprising a power supply circuit 21, a power input unit 22 and at least one charging area 23. The power supply circuit 21 comprises a first control board 210 electrically connected to the power input unit 22, and a microcontroller 211 installed on the first control board 210. The power input unit 22 is used to input the power provided by external power sources (such as city power supply, power supply device, etc.), comprising a power input socket 221, a power switch 222 and a circuit breaker 223. The power input socket 221 is connected to an external power supply through a power cord and is switched on/off by the power switch 222. The circuit breaker (non-fuse switch) 223 is used for overload protection of the input power. The charging area 23 is electrically connected to the first control board 210 of the power supply circuit 21, comprising at least one rack-mounted power distribution unit (PDU) 231 or power strips. Each power distribution unit 231 comprises a plurality of power sockets. Each charging area 23 can be driven by the power supply circuit 21 for power distribution, charging control and management functions.

The UV lamp set 3 is mounted in the housing space 10 of the housing 11 above the carrier rack 12, comprising a drive control circuit 31, at least one UV lamp 32, a start unit 33 and a power output unit 34. The drive control circuit 31 comprises a second circuit board 310 electrically connected to the first control board 210, and a microcontroller 311 and a power relay 312 or PCB relay installed on the second circuit board 310. The at least one UV lamp 32 is electrically connected to the second circuit board 310 through the start unit 33. Each UV lamp 32 comprises a lamp socket, and a UV lamp tube or lamp bulb installed on the lamp socket. The start unit 33 comprises an electronic ballast 331, a safety start switch (such as strip lamp/indicator lamp press button switch or knob switch) 332, at least one, for example, at least one door sensor switch (such as press button switches or infrared sensors) 333 that are connected in series. The electronic ballast 331 is electrically connected between the power relay 312 of the drive control circuit 31 and the plural UV lamps 32. The safety start switch 332 has the first and third contacts thereof electrically connected to the second circuit board 310 and the second and fourth contacts thereof electrically connected to the microcontroller 311 at the second circuit board 310, and also has the fourth contact thereof connected in parallel to the series of door sensor switches 333. The door sensor switch 333 is electrically connected the microcontroller 311 to form a start detection loop. The power output unit 34 is electrically connected to the second circuit board 310 of the drive control circuit 31, comprising a plurality of power sockets.

In the present preferred embodiment, the main body 1 of the smart charging device is a mobile charging cart. At least one mobile device 4 can be placed in each storage slot 121 of each carrier rack 12. Each mobile device 4 can include, but is not limited to, a smart phone, tablet, laptop, smart watch, smart glasses or other electronic devices that can be carried and worn on the body.

Each mobile device 4 can be electrically connected to any power socket of the power distribution unit 231 of any charging area 23 of the charging module 2 using a power transmission cable. Alternatively, each mobile device 4 can be directly connected to a connector module in the back side of any storage slot 121 of any carrier rack 12, and the connector module is then electrically connected to any power socket of the power distribution unit 231 of any charging area 23 of the charging module 2 using a power transmission cable. Thus, the overall space configuration of the storage mobile devices 4 is properly arranged, facilitating centralized management.

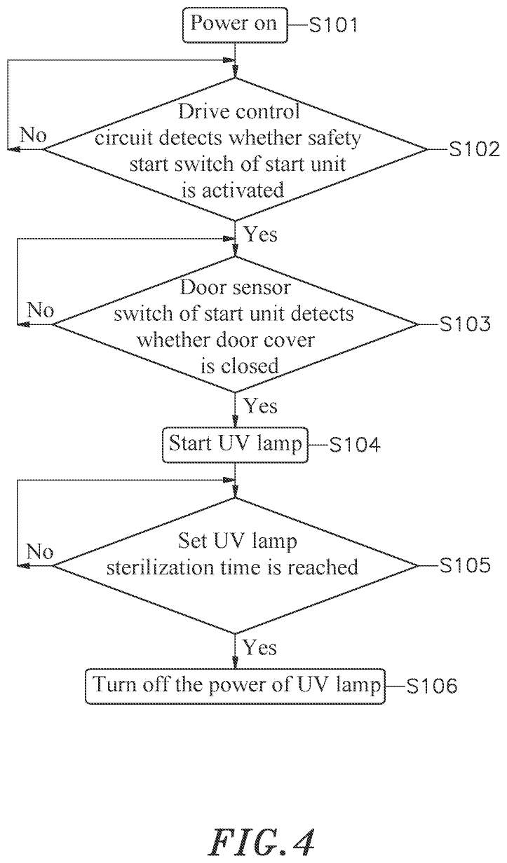

Referring to FIG. 4, the UV lamp control method applied to the smart charging device with UV lamp sterilization in accordance with the present invention comprises the steps of:

(S101) Power on.

(S102) The drive control circuit 31 detects whether the safety start switch 332 of the start unit 33 is activated? If yes, execute step (S103), if not, execute step (S102) repeatedly until the drive control circuit 31 detects that the safety start switch 332 is activated.

(S103) The door sensor switch 333 of the start unit 33 detects whether the door cover 13 is closed? If yes, execute step (S104), if not, execute step (S103) repeatedly until the door sensor switch 333 detects that the door cover 13 is closed.

(S104) Start UV lamps 32.

(S105) Determine whether the set UV lamp 32 sterilization time is reached? If yes, then execute step (S106), if not, then repeat step (S105) until the set sterilization time is reached.

(S106) Turn off the power of the UV lamps 32.

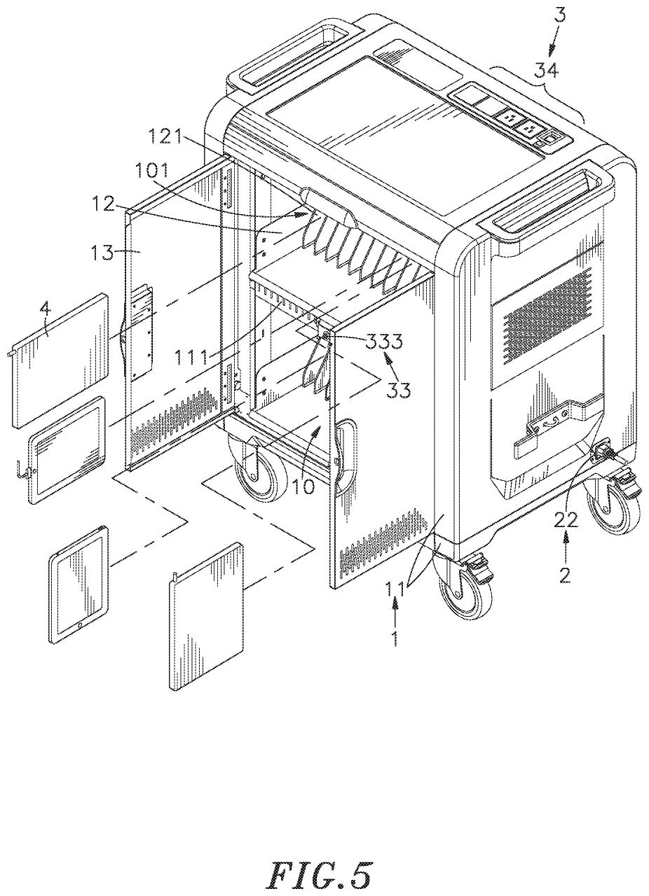

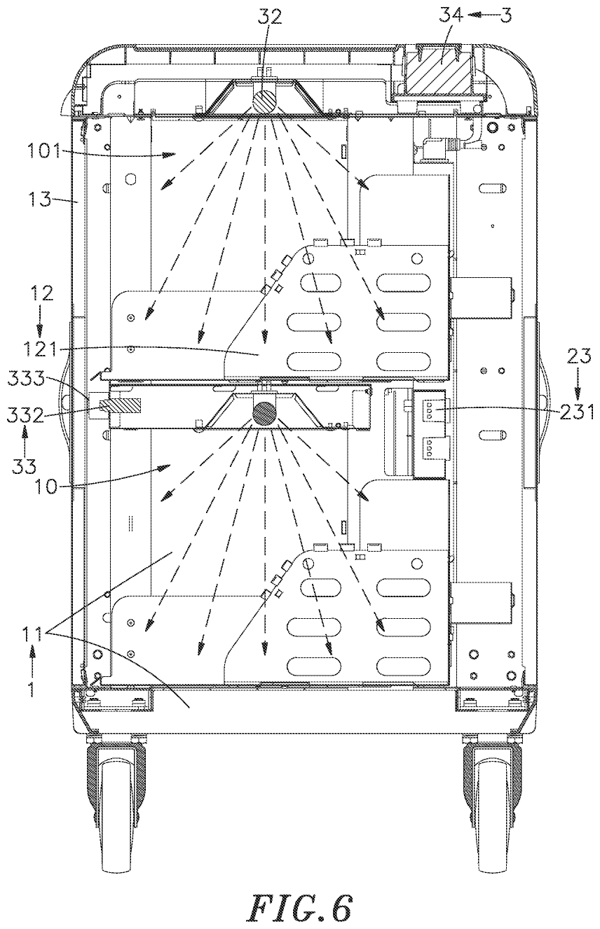

Referring to FIGS. 5 and 6, when the user presses the power switch 222 of the power input unit 22 to power on, the power supply circuit 21 directly provides the AC power input from the external power supply to each charging area 23, so that the power distribution unit 231 can quickly charge each mobile device 4 after the power transmission line plugged into the power socket thereof is converted into a stable direct current with a transformer. In an alternate form of the present invention, the power supply circuit 21 converts the AC power supply from the external power source into a stable direct current, and then the power distribution unit 231 of each charging area 23 charges the mobile devices 4 through a power transmission line or a connector module of the carrier rack 12. The charging areas 23 can also be controlled in turn through the power supply circuit 21 until each mobile device 4 is fully charged, so as to effectively increase the charging efficiency and shorten the charging time of each mobile device 4.

In the present preferred embodiment, the main body 1 of the smart charging device (such as a charging cart or a charging cabinet) not only has a charging function, but also has an ultraviolet sterilization function.

Mainly through the microcontroller 311 of the drive control circuit 31 to detect whether the safety start switch 332 of the start unit 33 has been activated by the user, and the indicator lamp on the lamp press button switch will light up after activation, representing the current sterilization function of the UV lamps 32 has entered the pre-start state. At this time, if the door cover 13 of the main body 1 is open, the door sensor switch 333 of the start unit 33 will not be activated, that is, the press button switch does not conduct and forms an open circuit state, so that the UV lamps 32 will not be activated. This is a mechanism to prevent users from accidentally activating the sterilization function to prevent the UV lamps 32 from being activated when the door cover 13 is open, which will cause damage to the human body due to UV leakage. The UV lamps 32 will be activated only after the door sensor switch 33 is activated, that is, the press button switch will turn on when the door cover 13 is closed. After the press button switch is switched on, the microcontroller 311 of the drive control circuit 31 will control the power relay 312 of the first control board 210 to provide power to the electronic ballast 331 for turning on the UV lamps 32 to emit ultraviolet light toward the mobile devices 4 in the carrier racks 12 to perform sterilization. At this point, the microcontroller 311 starts counting time. When the set sterilization time (such as three minutes) is reached, the power relay 312 will be controlled to turn off the power of the UV lamps 32 to complete the disinfection and sterilization of the mobile devices 4. It can effectively eliminate bacteria and viruses attached to the surface of mobile devices 4 in a short time, so that users are not likely to infect each other and cause harm to the human body during the process of picking or using mobile devices 4, better ensuring the safety of use.

Although a particular embodiment of the invention has been described in detail for purposes of illustration, various modifications and enhancements may be made without departing from the spirit and scope of the invention. Accordingly, the invention is not to be limited except as by the appended claims.

What the invention claimed is:

1. A smart charging device with UV lamp sterilization, comprising:
a main body comprising a housing, said housing comprising a housing space for the placement of at least one mobile device and a door covered installed on a front opening of said housing space;
a charging module comprising a power supply circuit, a power input unit electrically connected to said power supply circuit to input electricity provided by an external power supply and at least one charging area electrically connected to said power supply circuit for charging mobile devices; and
a UV lamp set comprising a drive control circuit electrically connected to said power supply circuit, a start unit and at least one UV lamp electrically connected to a microcontroller of said drive control circuit through said start unit, said start unit comprising an electronic ballast, a safety start switch and at least one door sensor switch electrically connected to said at least one said UV lamp;
wherein when said microcontroller detects that said safety start switch has been activated by a user, said at least one UV lamp enters the pre-start state, and when said at least one door sensor switch detects that said door cover is closed, said electronic ballast is controlled to start said at least one UV lamp to sterilize the mobile devices being placed in said housing space.

2. The smart charging device with UV lamp sterilization as claimed in claim 1, wherein said housing of said main body is selectively a rack or cabinet type, further comprising a plurality of partition plates arranged in said housing space at different elevations to divide said housing space into a plurality of vertically spaced storage chambers and a carrier rack mounted in each said storage chamber, each said carrier rack providing a plurality of transversely spaced storage slots for receiving the mobile devices.

3. The smart charging device with UV lamp sterilization as claimed in claim 1, wherein said power supply circuit of said charging module comprises a first control board electrically connected to said power input unit, and a microcontroller installed on said first control board; said charging area comprises a power distribution unit drivable by said microcontroller to charge each mobile device been placed in said housing space.

4. The smart charging device with UV lamp sterilization as claimed in claim 1, wherein said power input unit of said charging module comprises a power input socket connected to an external power supply for power input, a power switch for switching on/off said power input socket, and a circuit breaker used for overload protection of the input power.

5. The smart charging device with UV lamp sterilization as claimed in claim 1, wherein said drive control circuit of said UV lamp set comprises a second circuit board, and the microcontroller and a power relay installed on said second circuit board; said electronic ballast of said start unit is electrically connected between said power relay and said UV lamp so that the said microcontroller of said drive control circuit is capable of controlling said power relay to provide power supply to said electronic ballast to start said UV lamp.

6. A UV lamp control method applied to a smart charging device, said smart charging device comprising a main body and a charging module and a UV lamp set mounted in said smart charging device, said charging module comprising a power supply circuit, a power input unit electrically connected to said power supply circuit and at least one charging are for charging at least one mobile device been placed in said main body, said UV lamp set comprising a drive control circuit electrically connected to said power supply circuit, a start unit and at least one UV lamp electrically connected to said drive control circuit through said start unit for sterilizing the at least one mobile device been placed in said main body, the UV lamp control method comprising the steps of:
(A) powering on;
(B) said drive control circuit detecting whether a safety start switch of said start unit is activated, and then executing step (C) if said safety start switch of said start unit is activated, or executing step (B) repeatedly until said drive control circuit detects that said safety start switch is activated if said safety start switch of said start unit is not activated;
(C) a door sensor switch of said start unit detecting whether a door cover installed on an opening of said main body is closed, and then executing step (D) if said door cover is closed, or executing step (C) repeatedly until said door sensor switch detects that said door cover is closed if said door cover is not closed;
(D) starting said UV lamp;
(E) determining whether a preset UV lamp sterilization time is reached, and then executing step (F) if said preset UV lamp sterilization time is reached, or repeating step (E) until said set sterilization time is reached if said preset UV lamp sterilization time is not reached; and
(F) turning off the power of said UV lamp.

7. The UV lamp control method as claimed in claim 6, wherein said power supply circuit of said charging module comprises a microcontroller that controls an electronic ballast of said start unit to start said at least one UV lamp only when said safety start switch is activated by a user and said door sensor switch detects that said door cover is closed.

8. The UV lamp control method as claimed in claim 7, wherein in step (E), said microcontroller of said power supply circuit of said charging module counts said preset UV lamp sterilization time, and controls a drive control circuit to turn off the power of said at least one UV lamp when said preset UV lamp sterilization time is reached, and said preset UV lamp sterilization time is preferably 3 minutes.
